(19) United States
(12) Patent Application Publication
(10) Pub. No.: US 2025/0072848 A1
(43) Pub. Date: Mar. 6, 2025

(54) MEDICAL IMAGING SYSTEM

(71) Applicant: CANON KABUSHIKI KAISHA, Tokyo (JP)

(72) Inventors: YUKI NIWAMAE, Kanagawa (JP); TAKAHIRO YAMAMOTO, Kanagawa (JP); SEISHIRO TAKANO, Kanagawa (JP); SAKIKO TANIMOTO, Shizuoka (JP); SATOSHI SHINATA, Kanagawa (JP); TETSUYA SEKINE, Kanagawa (JP); SHOSAKU KAWASHIMA, Kanagawa (JP); NOBUO OSHIMOTO, Tokyo (JP); MEGUMI MATSUDA, Kanagawa (JP); YOSHINORI HIRANO, Chiba (JP)

(21) Appl. No.: 18/948,988

(22) Filed: Nov. 15, 2024

Related U.S. Application Data
(63) Continuation of application No. PCT/JP2023/018682, filed on May 19, 2023.

(30) Foreign Application Priority Data
May 26, 2022 (JP) 2022-085791

(51) Int. Cl. A61B 6/04; A61B 6/00

(52) U.S. Cl. CPC A61B 6/0487; A61B 6/4476

(57) ABSTRACT

To present, in advance, information about a movement direction corresponding to a predetermined operation unit of a console, a medical imaging system includes a bed 170 on which a subject is placed; a movement mechanism (bed movement mechanism 174) configured to move a table 158 on the bed 170 on the basis of operation information of a predetermined operation unit; a determining unit 118 configured to determine whether an operator has touched the predetermined operation unit; and a presenting unit (second presenting device 172) configured to present, around the bed 170 or on the table 158 on the bed 170, information about the movement direction corresponding to the predetermined operation unit when the determining unit 118 determines that the operator has touched the predetermined operation unit.

### CROSS-REFERENCE TO RELATED APPLICATIONS

This application is a Continuation of International Patent Application No. PCT/JP2023/018682, filed May 19, 2023, which claims the benefit of Japanese Patent Application No. 2022-085791, filed May 26, 2022, both of which are hereby incorporated by reference herein in their entirety.

### TECHNICAL FIELD

The present invention relates to a technique that presents, in a medical imaging system including a movement mechanism configured to move a component, such as a bed on which a subject is placed, information about movement of the component, such as a bed.

### BACKGROUND ART

There is a medical imaging system that includes a movement mechanism configured to move a component, such as a bed on which a subject is placed. A technique is disclosed in which a bed has an irradiating means configured to emit a marker beam onto a guide for connection to a gantry (see, e.g., Patent Literature 1).

### CITATION LIST

### Patent Literature

PTL 1 Japanese Patent Laid-Open No. 2004-329525

In the technique disclosed in Patent Literature 1, an operator brings the bed closer to a connection box for the gantry and presents, before connecting the bed, a marker beam by operating a switch. When the bed is moved with a predetermined operation unit (movement operation button) of a console, a direction indicated by the marker beam may not match a movement direction corresponding to the predetermined operation unit.

### SUMMARY OF INVENTION

An object of the present invention is to present, in advance, information about a movement direction corresponding to a predetermined operation unit of a console.

Note that the problem to be solved by embodiments disclosed in the present specification and drawings is not limited to that described above. Problems corresponding to the effect of each configuration described in the following embodiments can be positioned as other problems.

To achieve the object of the present invention, a medical imaging system includes a bed on which a subject is placed; a console including a plurality of operation units and configured to allow a predetermined operation unit to be operated to give an instruction to move a table on the bed in a movement direction corresponding to the predetermined operation unit; a movement mechanism configured to move the table on the bed on the basis of operation information of the predetermined operation unit; a determining unit configured to determine whether an operator has touched the predetermined operation unit; and a presenting unit configured to present, around the bed or on the table on the bed, information about the movement direction corresponding to the predetermined operation unit when the determining unit determines that the operator has touched the predetermined operation unit.

The medical imaging system may include a determining unit configured to determine whether an operator has pressed the predetermined operation unit halfway; and a presenting unit configured to present, around the bed or on the table on the bed, information about the movement direction corresponding to the predetermined operation unit when the determining unit determines that the operator has pressed the predetermined operation unit halfway.

Further features of the present invention will become apparent from the following description of exemplary embodiments with reference to the attached drawings.

### BRIEF DESCRIPTION OF DRAWINGS

FIG. 1A is an external view of a medical imaging system according to the present invention.

FIG. 1B is another external view of the medical imaging system according to the present invention.

FIG. 2 is a diagram illustrating an exemplary console of the medical imaging system according to the present invention.

FIG. 3 is a diagram illustrating an exemplary configuration of Embodiment 1 of the medical imaging system according to the present invention.

FIG. 4A is a diagram illustrating a mode of presentation related to a bed of the medical imaging system according to the present invention.

FIG. 4B is a diagram illustrating a mode of presentation related to the bed of the medical imaging system according to the present invention.

FIG. 4C is a diagram illustrating a mode of presentation related to the bed of the medical imaging system according to the present invention.

FIG. 4D is a diagram illustrating a mode of presentation related to the bed of the medical imaging system according to the present invention.

FIG. 5A is a diagram illustrating a mode of presentation related to the bed of the medical imaging system according to the present invention.

FIG. 5B is a diagram illustrating a mode of presentation related to the bed of the medical imaging system according to the present invention.

FIG. 5C is a diagram illustrating a mode of presentation related to the bed of the medical imaging system according to the present invention.

FIG. 5D is a diagram illustrating a mode of presentation related to the bed of the medical imaging system according to the present invention.

FIG. 6A is a diagram illustrating a mode of presentation related to the bed of the medical imaging system according to the present invention.

FIG. 6B is a diagram illustrating a mode of presentation related to the bed of the medical imaging system according to the present invention.

FIG. 6C is a diagram illustrating a mode of presentation related to the bed of the medical imaging system according to the present invention.

FIG. 7A is a diagram illustrating a mode of presentation related to a radiation detecting unit of the medical imaging system according to the present invention.

FIG. 7B is a diagram illustrating a mode of presentation related to the radiation detecting unit of the medical imaging system according to the present invention.

FIG. 7C is a diagram illustrating a mode of presentation related to the radiation detecting unit of the medical imaging system according to the present invention.

FIG. 7D is a diagram illustrating a mode of presentation related to the radiation detecting unit of the medical imaging system according to the present invention.

FIG. 8 is a flowchart illustrating an operation of the medical imaging system according to the present invention.

FIG. 9 is an external view of the medical imaging system according to the present invention.

FIG. 10A is a diagram illustrating a mode of presentation related to the bed of the medical imaging system according to the present invention.

FIG. 10B is another diagram illustrating the mode of presentation related to the bed of the medical imaging system according to the present invention.

FIG. 11 is a diagram illustrating an exemplary configuration of Embodiment 2 of the medical imaging system according to the present invention.

FIG. 12A is a diagram illustrating a mode of presentation related to the bed of the medical imaging system according to the present invention.

FIG. 12B is a diagram illustrating a mode of presentation related to the bed of the medical imaging system according to the present invention.

FIG. 12C is a diagram illustrating a mode of presentation related to the bed of the medical imaging system according to the present invention.

FIG. 13A is a diagram illustrating a mode of presentation related to the bed of the medical imaging system according to the present invention.

FIG. 13B is a diagram illustrating a mode of presentation related to the bed of the medical imaging system according to the present invention.

FIG. 13C is a diagram illustrating a mode of presentation related to the bed of the medical imaging system according to the present invention.

FIG. 14A is a diagram illustrating a mode of presentation related to the bed of the medical imaging system according to the present invention.

FIG. 14B is a diagram illustrating a mode of presentation related to the bed of the medical imaging system according to the present invention.

FIG. 14C is a diagram illustrating a mode of presentation related to the bed of the medical imaging system according to the present invention.

### DESCRIPTION OF EMBODIMENTS

Preferred embodiments of the present invention will now be described with reference to the accompanying drawings.

### Embodiment 1

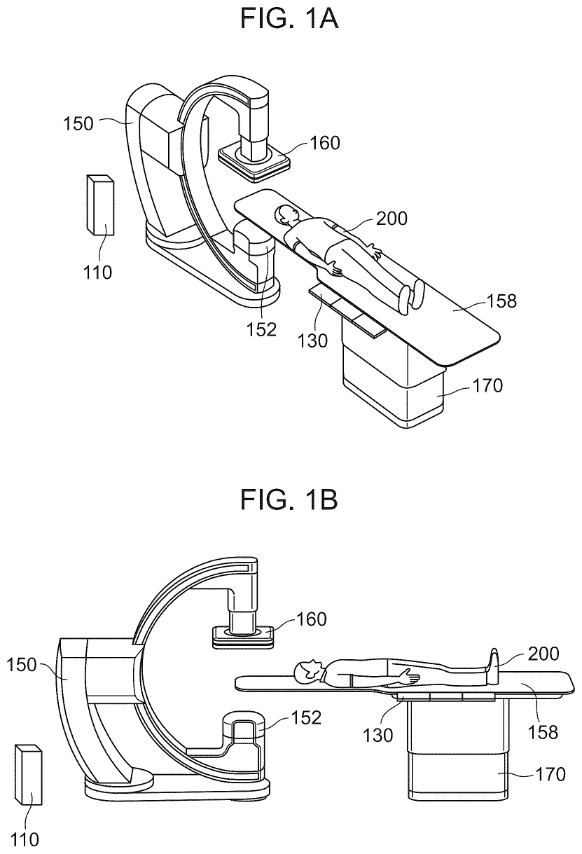

Embodiment 1 of a medical imaging system according to the present invention will be described using FIG. 1A to FIG. 8. FIG. 1A and FIG. 1B are external views of the medical imaging system according to the present embodiment. FIG. 1A is a perspective view and FIG. 1B is a lateral view. As illustrated in FIG. 1A and FIG. 1B, the medical imaging system of the present invention includes a controller 110, a console 130, an imager 150, and a bed 170.

The controller 110 has the function of controlling each component of the medical imaging system. The controller 110 is connected to each of the components, such as the console 130, the imager 150, and the bed 170. Therefore, the controller 110 can control each of the components, such as the imager 150 and the bed 170, on the basis of operation instructions from the console 130. The controller 110 may be installed inside the imager 150 or the bed 170, or may be installed outside the imaging room.

The medical imaging system includes the imager 150 with a C-arm. The imager 150 includes a radiation irradiation unit 152 and a radiation detecting unit 160. The C-arm supports the radiation irradiation unit 152 and the radiation detecting unit 160. The imager 150 includes an imager movement mechanism configured to move the radiation irradiation unit 152 and the radiation detecting unit 160 through the C-arm. The imager movement mechanism can rotate the C-arm, and can rotate and move the radiation irradiation unit 152 and the radiation detecting unit 160.

The imager movement mechanism can vertically move the radiation detecting unit 160. Also, the imager movement mechanism can horizontally slide the radiation detecting unit 160.

The radiation irradiation unit 152 has the function of generating radiation. The radiation irradiation unit 152 is implemented, for example, by an X-ray tube and emits radiation toward a subject 200 (e.g., a specific part of the subject).

The radiation detecting unit 160 has the function of detecting radiation emitted from the radiation irradiation unit 152. The radiation detecting unit 160 detects radiation transmitted through the subject 200 and outputs image data corresponding to the radiation. The image data can also be referred to as a radiation image. Specifically, the radiation detecting unit 160 detects radiation transmitted through the subject 200 as electric charge equivalent to the amount of transmitted radiation. Examples of the radiation detecting unit 160 include a direct conversion type sensor that directly converts radiation into electric charge, such as a-Se that converts radiation into electric charge, and an indirect type sensor using a scintillator, such as CsI, and a photoelectric conversion element, such as a-Si. The radiation detecting unit 160 A/D converts the detected electric charge to generate image data and outputs the image data to the controller 110.

The radiation irradiation unit 152 is a vacuum tube that generates radiation by emitting thermal electrons from a cathode (filament) toward an anode (target) through application of a high voltage from a radiation high-voltage device and supply of filament current. Radiation is generated by collision of thermal electrons with the target.

The radiation irradiation unit 152 has a radiation aperture that blocks radiation. The radiation aperture is provided in front of a radiation emission frame in the radiation irradiation unit 152. The radiation aperture has, for example, four aperture blades formed by metal plates made of lead plates. The aperture blades are set in accordance with the region of interest entered by the operator through the console 130. A driving device slides the aperture blades to allow the radiation aperture to adjust the region for blocking radiation to any size.

Radiation generated by the radiation irradiation unit 152 can be narrowed down such that the region of interest of the subject 200 can be irradiated.

The medical imaging system includes the bed 170 on which the subject 200 is placed. A table 158 is disposed on the upper surface of the bed 170. The bed 170 includes a bed movement mechanism configured to move the table 158. The bed movement mechanism can horizontally and vertically move the table 158.

Rails (not illustrated) are disposed on the lower surface of the table 158. For example, the rails are disposed in the long axis direction and the short axis direction of the table 158. The table 158 can slide (or horizontally move) along the long axis direction and the short axis direction of the table 158.

The medical imaging system includes the console 130. The console 130 may be installed outside the imaging room. The console 130 includes a plurality of operation units. Operating the operation units on the console 130 can give movement instructions to the controller 110 configured to control the imager movement mechanism, the bed movement mechanism, and the like. By operating the console 130 and controlling the controller 110, the imager movement mechanism and the bed movement mechanism can move the radiation irradiation unit 152, the radiation detecting unit 160, and the table 158 on the bed 170 in a desired direction. The imager movement mechanism and the bed movement mechanism can implement the movement through, for example, a motor or an actuator (not illustrated).

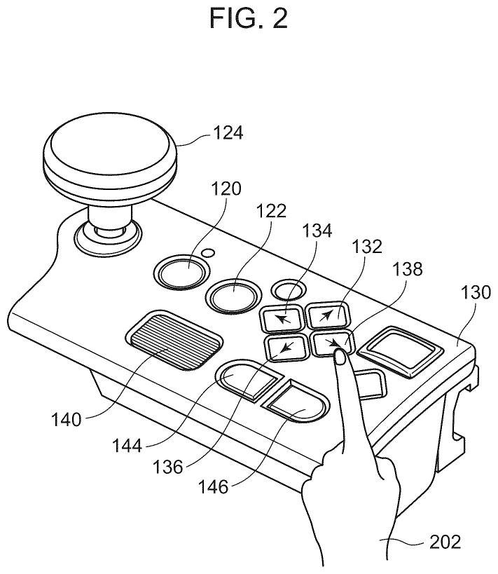

FIG. 2 is a diagram illustrating an example of the console 130 of the medical imaging system according to the present invention. The console 130 includes a plurality of operation units. The plurality of operation units of the console 130 are connected to the controller 110 and can give instructions to the controller 110. The controller 110 controls each component in accordance with instructions from the plurality of operation units.

The console 130 includes, for example, a first operation unit 132, a second operation unit 134, a third operation unit 136, a fourth operation unit 138, and an irradiation operation unit 140. The plurality of operation units include buttons and a touch panel.

The first operation unit 132, the second operation unit 134, the third operation unit 136, and the fourth operation unit 138 are operation units for operation of horizontally moving the table 158 on the bed 170. A fifth operation unit 144 and a sixth operation unit 146 are operation units for operation of vertically moving the table 158 on the bed 170.

Switching operation units 120 and 122 are operation units for switching the operation target. For example, the operation target can be switched from the table 158 on the bed 170 to the imager 150.

A stick operation unit 124 has the functions of the first operation unit 132, the second operation unit 134, the third operation unit 136, and the fourth operation unit 138. The stick operation unit 124 allows the operation of horizontally moving the table 158 on the bed 170.

Each operation unit can distinguish between a state in which an operator 202 has touched the operation unit and a state in which the operator 202 has fully pressed the operation unit. Each operation unit can also distinguish between a state in which the operator 202 has pressed the operation unit halfway and a state in which the operator 202 has fully pressed the operation unit.

For example, each operation unit includes a contact sensor that detects a touch by the operator 202. Each operation unit also includes a press sensor that detects a state in which the operator 202 has fully pressed the operation unit.

The contact sensor is installed on the surface of each operation unit. The contact sensor may be included inside each operation unit. When the operator 202 presses an operation unit (button), the contact sensor detects a touch by a fingertip of the operator 202. The operation unit notifies the controller 110 that the touch by the operator 202 has been detected. Examples of the contact sensor include a capacitive sensor and a pressure sensor.

The capacitive sensor is a non-contact type sensor using an electric field, and can detect the presence of a touch by an object. When an object touches the operation unit, that is, when an object enters the electric field, a change in capacitance allows detection of the object. A change in capacitance detected by the capacitive sensor makes it possible to detect a touch by the operator 202.

The pressure sensor is a contact-type sensor using pressure, and can detect the presence of a touch by an object. When an object touches the operation unit, the touch by the object changes pressure. A change in pressure detected by the pressure sensor makes it possible to detect a touch by the operator 202.

Although a capacitive sensor and a pressure sensor have been described as examples of the contact sensor, the type of the contact sensor is not limited to those described above. As described above, each operation unit can distinguish between a state in which the operator 202 has touched the operation unit and a state in which the operator 202 has fully pressed the operation unit.

When the operator 202 fully presses the operation unit while touching the operation unit, the operation unit can detect that it has been fully pressed. The operation unit can notify the controller 110 of detection of been fully pressed by the operator 202.

Each operation unit may be of a dual press type, including a half press and a full press. A half press is a state in which the operation unit (button) is not fully pressed but is pressed halfway. A full press is a state in which the operation unit (button) is fully pressed.

Each operation unit includes a press sensor configured to detect a state of being pressed halfway by the operator 202 and a state of being fully pressed by the operator 202. When a press (pressure) corresponding to a half press is detected, the operation unit transmits the detection of a half press to the controller 110, whereas when a press (pressure) corresponding to a full press is detected, the operation unit transmits the detection of a full press to the controller 110. The stick operation unit 124 may similarly be of a dual press type, including a half press and a full press. The stick operation unit 124 includes a sensor configured to detect a state of being pressed halfway by the operator 202 and a state of being fully pressed by the operator 202. It is thus possible to distinguish between a state in which the operator 202 has pressed the operation unit halfway and a state in which the operator 202 has fully pressed the operation unit.

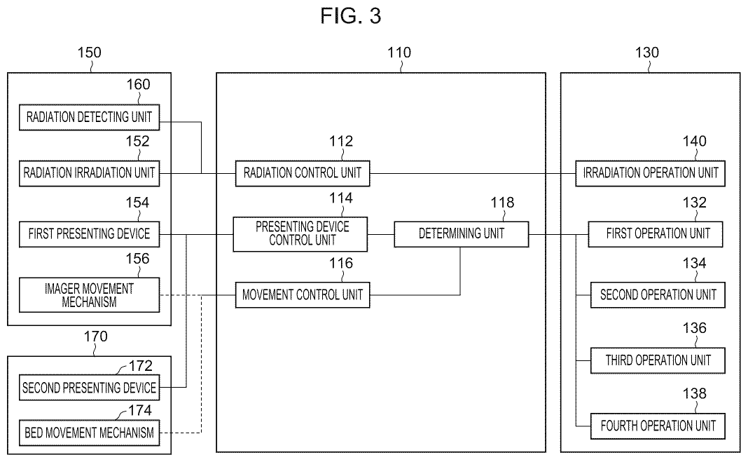

FIG. 3 is a diagram illustrating an exemplary configuration of the medical imaging system according to the present invention. The controller 110 includes a radiation control unit 112, a presenting device control unit 114, a movement control unit 116, and a determining unit 118.

The radiation control unit 112 has the function of controlling the radiation irradiation unit 152 in the imager 150. The radiation control unit 112 sets imaging conditions (tube voltage, tube current, and irradiation time) for radiation emitted from the radiation irradiation unit 152. The radiation control unit 112 can control the timing of irradiation by the radiation irradiation unit 152 and the timing of imaging by the radiation detecting unit 160. The radiation control unit 112 can cause the radiation irradiation unit 152 to emit radiation multiple times, and the radiation detecting unit 160 can perform imaging multiple times in accordance with emission from the radiation irradiation unit 152.

The presenting device control unit 114 has the function of controlling presenting devices configured to perform presentation about the movement directions of the imager 150 and the bed 170. The movement control unit 116 has the function of controlling movement of the imager 150 and the bed 170. The determining unit 118 has the function of determining an operation state of each operation unit on the console 130. The operation state refers to, for example, a state in which the operator has touched the operation unit, a state in which the operator has pressed the operation unit halfway, or a state in which the operator has fully pressed the operation unit.

The console 130 includes a plurality of operation units. The console 130 includes, for example, the first operation unit 132, the second operation unit 134, the third operation unit 136, the fourth operation unit 138, and the irradiation operation unit 140. With the first operation unit 132, the second operation unit 134, the third operation unit 136, and the fourth operation unit 138, the table 158 on the bed 170 can be moved forward, backward, rightward, and leftward. Although the console 130 includes four operation units here, the console 130 may include more than four operation units.

To simplify the explanation, the illustrations of components other than the first operation unit 132, the second operation unit 134, the third operation unit 136, the fourth operation unit 138, and the irradiation operation unit 140 of the console 130 are omitted. For example, the fifth operation unit 144, the sixth operation unit 146, and the stick operation unit 124 for vertically moving the table 158 on the bed 170 may also be illustrated.

As illustrated in FIG. 2 and FIG. 3, the first operation unit 132 is an operation unit for operation of moving, for example, the table 158 on the bed 170 rightward. The second operation unit 134 is an operation unit for operation of moving, for example, the table 158 on the bed 170 forward. The third operation unit 136 is an operation unit for operation of moving, for example, the table 158 on the bed 170 leftward. The fourth operation unit 138 is an operation unit for operation of moving, for example, the table 158 on the bed 170 backward. The irradiation operation unit 140 is an operation unit for operation related to radiation irradiation. The irradiation operation unit 140 has the function of changing irradiation conditions and starting irradiation.

The imager 150 includes the radiation irradiation unit 152, the radiation detecting unit 160, a first presenting device 154, and an imager movement mechanism 156.

The radiation irradiation unit 152 can emit radiation in a desired range, as described above. The radiation irradiation unit 152 is installed on the C-arm installed on the floor surface or ceiling.

As described above, the radiation detecting unit 160 can detect radiation emitted from the radiation irradiation unit 152. The radiation detecting unit 160 is installed on the C-arm to face the radiation irradiation unit 152.

The first presenting device 154 has the function of presenting the movement direction of the imager 150. When an operation of moving the imager 150 forward has been performed on the console 130, the first presenting device 154 presents, on or around the imager 150, information indicating that the imager 150 is being moved forward. When an operation of moving the imager 150 backward has been performed on the console 130, the first presenting device 154 presents, on or around the imager 150, information indicating that the imager 150 is being moved backward.

Specifically, the determining unit 118 determines whether the operator has touched or pressed halfway the operation unit corresponding to an operation of moving the radiation detecting unit 160 of the imager 150 clockwise. When the determining unit 118 determines that the operator has touched or pressed halfway the operation unit corresponding to an operation of moving the radiation detecting unit 160 of the imager 150 clockwise, the presenting device control unit 114 controls the first presenting device 154 to present, on or around the radiation detecting unit 160, information indicating that the radiation detecting unit 160 is to be moved clockwise (i.e., information indicating that clockwise movement is scheduled). Then when the determining unit 118 determines that the operator has fully pressed the operation unit, the first presenting device 154 presents, on or around the radiation detecting unit 160, information indicating that the radiation detecting unit 160 is being moved clockwise (i.e., information indicating that clockwise movement is in progress). The movement control unit 116 controls the imager movement mechanism 156 to move the radiation detecting unit 160 clockwise.

The determining unit 118 determines whether the operator has touched or pressed halfway the operation unit corresponding to an operation of moving the radiation detecting unit 160 of the imager 150 counterclockwise. When the determining unit 118 determines that the operator has touched or pressed halfway the operation unit corresponding to an operation of moving the radiation detecting unit 160 of the imager 150 counterclockwise, the presenting device control unit 114 controls the first presenting device 154 to present, on or around the radiation detecting unit 160, information indicating that the radiation detecting unit 160 is to be moved counterclockwise (i.e., information indicating that counterclockwise movement is scheduled). Then when the determining unit 118 determines that the operator has fully pressed the operation unit, the first presenting device 154 presents, on or around the radiation detecting unit 160, information indicating that the radiation detecting unit 160 is being moved counterclockwise (i.e., information indicating that counterclockwise movement is in progress). The movement control unit 116 controls the imager movement mechanism 156 to move the radiation detecting unit 160 counterclockwise.

The determining unit 118 determines whether the operator has touched or pressed halfway the operation unit corresponding to an operation of moving the radiation detecting unit 160 of the imager 150 downward. When the determining unit 118 determines that the operator has touched or pressed halfway the operation unit corresponding to an operation of moving the radiation detecting unit 160 of the imager 150 downward, the presenting device control unit 114 controls the first presenting device 154 to present, on or around the radiation detecting unit 160, information indicating that the radiation detecting unit 160 is to be moved downward (i.e., information indicating that downward movement is scheduled). Then when the determining unit 118 determines that the operator has fully pressed the operation unit, the first presenting device 154 presents, on or around the radiation detecting unit 160, information indicating that the radiation detecting unit 160 is being moved downward (i.e., information indicating that downward movement is in progress). The movement control unit 116 controls the imager movement mechanism 156 to move the radiation detecting unit 160 downward.

The first operation unit 132, the second operation unit 134, the third operation unit 136, and the fourth operation unit 138 can distinguish between a state in which the operator has touched the operation unit, a state in which the operator has pressed the operation unit halfway, and a state in which the operator has fully pressed the operation unit.

The second presenting device 172 in the bed 170 has the function of presenting the movement direction of the table 158 on the bed 170. Specifically, the determining unit 118 determines whether the operator has touched or pressed halfway the second operation unit 134 corresponding to an operation of moving the table 158 on the bed 170 forward. When the determining unit 118 determines that the operator has touched or pressed halfway the second operation unit 134 corresponding to an operation of moving the table 158 on the bed 170 forward, the presenting device control unit 114 controls the second presenting device 172 to present, around the bed 170 or on the table 158 on the bed 170, information indicating that the table 158 on the bed 170 is to be moved forward (i.e., information indicating that forward movement is scheduled). Note that “around the bed 170” refers to a region near the bed 170, such as a floor surface within 1 m of the bed 170.

Then when the determining unit 118 determines that the operator has fully pressed the second operation unit 134, the second presenting device 172 presents, around the bed 170 or on the table 158 on the bed 170, information indicating that the table 158 on the bed 170 is being moved forward (i.e., information indicating that forward movement is in progress). Also, when the determining unit 118 determines that the operator has fully pressed the second operation unit 134, the movement control unit 116 controls the bed movement mechanism 174 to move the table 158 on the bed 170 forward.

The determining unit 118 also determines whether the operator has touched or pressed halfway the fourth operation unit 138 corresponding to an operation of moving the table 158 on the bed 170 backward. When the determining unit 118 determines that the operator has touched or pressed halfway the fourth operation unit 138 corresponding to an operation of moving the table 158 on the bed 170 backward, the presenting device control unit 114 controls the second presenting device 172 to present, around the bed 170 or on the table 158 on the bed 170, information indicating that the table 158 on the bed 170 is to be moved backward (i.e., information indicating that backward movement is scheduled). Then when the determining unit 118 determines that the operator has fully pressed the fourth operation unit 138, the second presenting device 172 presents, around the bed 170 or on the table 158 on the bed 170, information indicating that the table 158 on the bed 170 is being moved backward (i.e., information indicating that backward movement is in progress). Also, when the determining unit 118 determines that the operator has fully pressed the fourth operation unit 138, the movement control unit 116 controls the bed movement mechanism 174 to move the table 158 on the bed 170 backward.

The determining unit 118 also determines whether the operator has touched or pressed halfway the first operation unit 132 corresponding to an operation of moving the table 158 on the bed 170 rightward. When the determining unit 118 determines that the operator has touched or pressed halfway the first operation unit 132 corresponding to an operation of moving the table 158 on the bed 170 rightward, the presenting device control unit 114 controls the second presenting device 172 to present, around the bed 170 or on the table 158 on the bed 170, information indicating that the table 158 on the bed 170 is to be moved rightward (i.e., information indicating that rightward movement is scheduled). Then when the determining unit 118 determines that the operator has fully pressed the first operation unit 132, the second presenting device 172 presents, around the bed 170 or on the table 158 on the bed 170, information indicating that the table 158 on the bed 170 is being moved rightward (i.e., information indicating that rightward movement is in progress). Also, when the determining unit 118 determines that the operator has fully pressed the first operation unit 132, the movement control unit 116 controls the bed movement mechanism 174 to move the table 158 on the bed 170 rightward. The description of the third operation unit 136 will be omitted, as the third operation unit 136 is the same as the first operation unit 132 except that it corresponds to leftward movement.

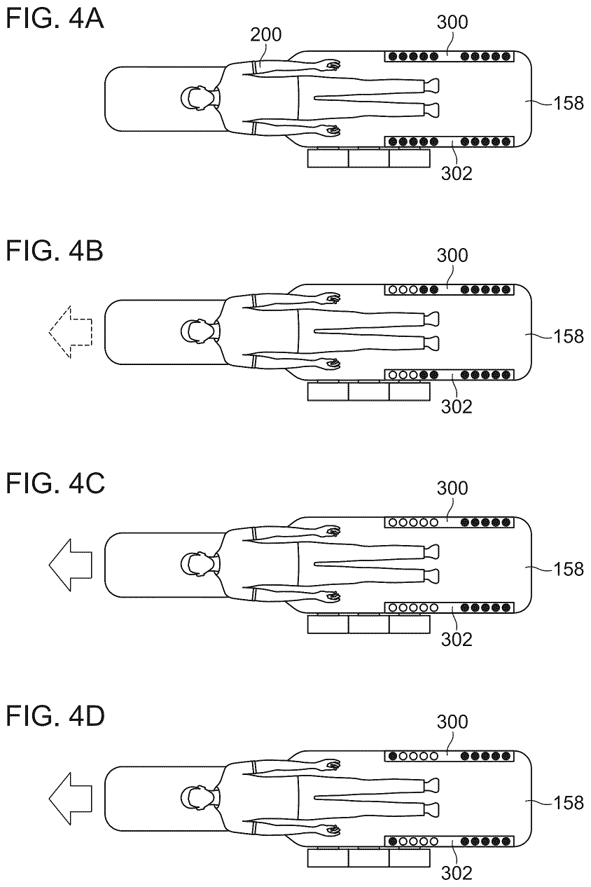

FIG. 4A to FIG. 7D are diagrams each illustrating an exemplary mode of presentation of the medical imaging system according to the present invention. FIG. 4A to FIG. 6C are diagrams each illustrating a mode of presentation related to the bed 170 of the medical imaging system according to the present invention. As illustrated in FIG. 4A to FIG. 6C, a plurality of light-emitting devices (LED) 300 and 302 are installed at both ends of the table 158 on the bed 170. The plurality of light-emitting devices 300 and 302 correspond to the second presenting device 172 illustrated in FIG. 3, and the second presenting device 172 includes the plurality of light-emitting devices 300 and 302. Specifically, the plurality of light-emitting devices 300 and 302 are installed at both ends of the table 158, with the subject 200 therebetween. The plurality of light-emitting devices 300 and 302 are arranged in multiple rows at both ends of the table 158. The plurality of light-emitting devices 300 and 302 are arranged along the long axis direction of the table 158. Here, ten light-emitting devices 300 and ten light-emitting devices 302 each are arranged long the long axis direction of the table 158. Light-emitting devices in black are in a non-light-emitting state, and light-emitting devices in white are in a light-emitting state (in a lit state).

FIG. 4A to FIG. 4D each illustrate a mode in which the table 158 on the bed 170 moves forward. Moving forward means that the table 158 on the bed 170 moves leftward in the drawing (or toward the head of the subject 200).

FIG. 4A illustrates a state in which the operator is not operating (or touching) any operation unit of the console 130 and the table 158 on the bed 170 is at rest. At this point, the plurality of light-emitting devices 300 and 302 are not emitting light.

FIG. 4B illustrates a state in which the operator has touched the second operation unit 134 of the console 130 or the operator has pressed halfway the second operation unit 134 of the console 130. The second operation unit 134 corresponds to an operation of moving the table 158 on the bed 170 forward.

When the determining unit 118 determines that the operator has touched or pressed halfway the second operation unit 134, the presenting device control unit 114 presents, through the plurality of light-emitting devices 300 and 302 (second presenting device 172), information indicating that the table 158 on the bed 170 is to be moved forward (i.e., information indicating that forward movement is scheduled). The presenting device control unit 114 controls the plurality of light-emitting devices 300 and 302 such that light-emitting devices corresponding to the forward direction emit light. For example, of the plurality of light-emitting devices 300 and 302, six light-emitting devices corresponding to the forward direction (or on the left side in the drawing) emit light. That is, the presenting device control unit 114 performs control such that the light-emitting devices corresponding to the movement direction emit light.

By operating the second operation unit 134 as described above, the operator can recognize, without fully pressing the second operation unit 134, that the table 158 on the bed 170 is to move forward.

FIG. 4C illustrates a state in which the operator has fully pressed the second operation unit 134 of the console 130. When the determining unit 118 determines that the operator has fully pressed the second operation unit 134, the presenting device control unit 114 presents, through the plurality of light-emitting devices 300 and 302, information indicating that the table 158 on the bed 170 is being moved forward (i.e., information indicating that forward movement is in progress). The presenting device control unit 114 controls the plurality of light-emitting devices 300 and 302 such that light-emitting devices corresponding to the forward direction emit light.

The number of light-emitting devices that emit light when the determining unit 118 determines that the operator has fully pressed the second operation unit 134 is greater than the number of light-emitting devices that emit light when the determining unit 118 determines that the operator has touched or pressed halfway the second operation unit 134. For example, of the plurality of light-emitting devices 300 and 302, ten light-emitting devices corresponding to the forward direction (or on the left side in the drawing) emit light.

In the description above, the number of light-emitting devices 300 and 302 emitting light is used to distinguish between information indicating that the table 158 on the bed 170 is to be moved forward (i.e., information indicating that forward movement is scheduled) and information indicating that the table 158 on the bed 170 is being moved forward (i.e., information indicating that forward movement is in progress). It is simply required that a plurality of light-emitting devices (second presenting device 172) emitting light when the determining unit 118 determines that the operator has touched or pressed halfway the second operation unit 134 and a plurality of light-emitting devices (second presenting device 172) emitting light when the determining unit 118 determines that the operator has fully pressed the second operation unit 134 have different modes of presentation.

Thus, when fully pressing the second operation unit 134, the operator can recognize that the table 158 on the bed 170 is moving forward (i.e., forward movement is in progress).

FIG. 4D illustrates a state in which the operator has fully pressed the second operation unit 134 of the console 130, and indicates that the table 158 on the bed 170 is near the limit of forward movement. The limit of movement is set by the movable range of the table 158 on the bed 170 (e.g., rail length). The presenting device control unit 114 presents, through the plurality of light-emitting devices 300 and 302, information indicating that the table 158 on the bed 170 is being moved forward and is near the limit of movement (i.e., information indicating that forward movement is in progress and the limit of the movement is near). The presenting device control unit 114 controls the plurality of light-emitting devices 300 and 302 such that at least one of the plurality of light-emitting devices configured to emit light corresponding to the forward direction does not emit light.

For example, of the plurality of light-emitting devices 300 and 302, eight light-emitting devices corresponding to the forward direction (or on the left side in the drawing) emit light, but the light-emitting device at the leftmost end is not emitting light. By checking that the light-emitting device at the leftmost end is not emitting light, the operator can recognize that the table 158 on the bed 170 is being moved forward and that the limit of movement is near.

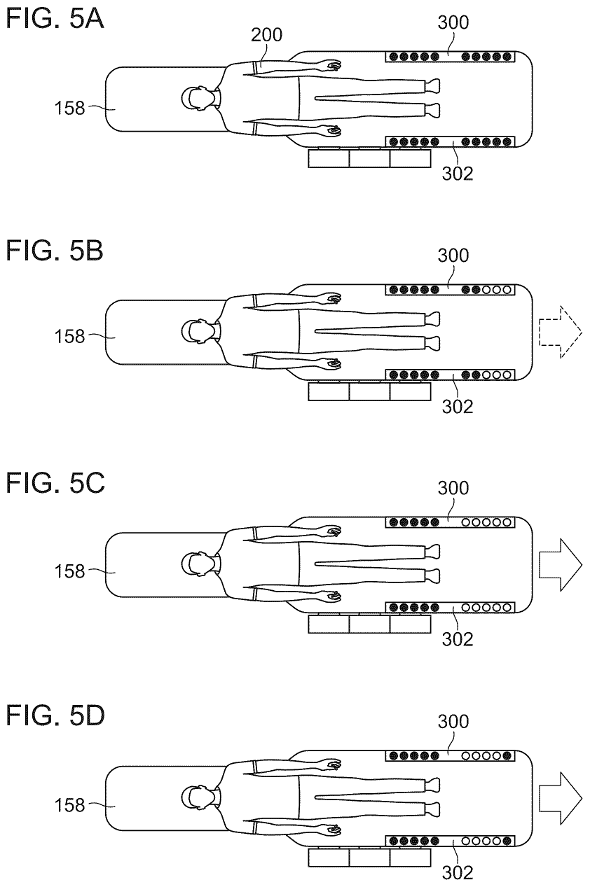

FIG. 5A to FIG. 5D each illustrate a mode in which the table 158 on the bed 170 moves backward. Moving backward means that the table 158 on the bed 170 moves rightward in the drawing (or in the direction of the feet of the subject 200).

FIG. 5A illustrates a state in which the operator is not operating (or touching) any operation unit of the console 130 and the table 158 on the bed 170 is at rest. At this point, the plurality of light-emitting devices 300 and 302 are not emitting light.

FIG. 5B illustrates a state in which the operator has touched the fourth operation unit 138 of the console 130 or the operator has pressed halfway the fourth operation unit 138 of the console 130. The fourth operation unit 138 corresponds to an operation of moving the table 158 on the bed 170 backward.

When the determining unit 118 determines that the operator has touched or pressed halfway the fourth operation unit 138, the presenting device control unit 114 presents, through the plurality of light-emitting devices 300 and 302 (second presenting device 172), information indicating that the table 158 on the bed 170 is to be moved backward (i.e., information indicating that backward movement is scheduled). The presenting device control unit 114 controls the plurality of light-emitting devices 300 and 302 such that light-emitting devices corresponding to the backward direction emit light. For example, of the plurality of light-emitting devices 300 and 302, six light-emitting devices corresponding to the backward direction (or on the right side in the drawing) emit light.

By operating the fourth operation unit 138 as described above, the operator can recognize, without fully pressing the fourth operation unit 138, that the table 158 on the bed 170 is to move backward.

FIG. 5C illustrates a state in which the operator has fully pressed the fourth operation unit 138 of the console 130. When the determining unit 118 determines that the operator has fully pressed the fourth operation unit 138, the presenting device control unit 114 presents, through the plurality of light-emitting devices 300 and 302, information indicating that the table 158 on the bed 170 is being moved backward (i.e., information indicating that backward movement is in progress). The presenting device control unit 114 controls the plurality of light-emitting devices 300 and 302 such that light-emitting devices corresponding to the backward direction emit light.

The number of light-emitting devices that emit light when the determining unit 118 determines that the operator has fully pressed the fourth operation unit 138 is greater than the number of light-emitting devices that emit light when the determining unit 118 determines that the operator has touched or pressed halfway the fourth operation unit 138. For example, of the plurality of light-emitting devices 300 and 302, ten light-emitting devices corresponding to the backward direction (or on the right side in the drawing) emit light.

In the description above, the number of light-emitting devices 300 and 302 emitting light is used to distinguish between information indicating that the table 158 on the bed 170 is to be moved backward (i.e., information indicating that backward movement is scheduled) and information indicating that the table 158 on the bed 170 is being moved backward (i.e., information indicating that backward movement is in progress). It is simply required that a plurality of light-emitting devices (second presenting device 172) emitting light when the determining unit 118 determines that the operator has touched or pressed halfway the fourth operation unit 138 and a plurality of light-emitting devices (second presenting device 172) emitting light when the determining unit 118 determines that the operator has fully pressed the fourth operation unit 138 have different modes of presentation.

Thus, when fully pressing the fourth operation unit 138, the operator can recognize that the table 158 on the bed 170 is moving backward (i.e., backward movement is in progress).

FIG. 5D illustrates a state in which the operator has fully pressed the fourth operation unit 138 of the console 13, and indicates that the table 158 on the bed 170 is near the limit of backward movement. The presenting device control unit 114 presents, through the plurality of light-emitting devices 300 and 302, information indicating that the table 158 on the bed 170 is being moved backward and is near the limit of movement (i.e., information indicating that backward movement is in progress and the limit of the movement is near). The presenting device control unit 114 controls the plurality of light-emitting devices 300 and 302 such that at least one of the plurality of light-emitting devices configured to emit light corresponding to the backward direction does not emit light.

For example, of the plurality of light-emitting devices 300 and 302, eight light-emitting devices corresponding to the backward direction (or on the right side in the drawing) emit light, but the light-emitting device at the rightmost end is not emitting light. By checking that the light-emitting device at the rightmost end is not emitting light, the operator can recognize that the table 158 on the bed 170 is being moved backward and that the limit of movement is near.

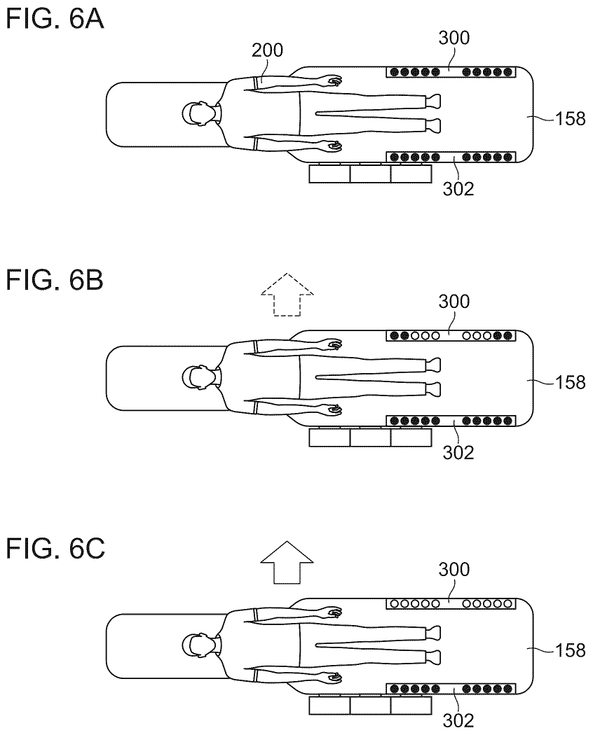

FIG. 6A to FIG. 6C each illustrate a mode in which the table 158 on the bed 170 moves rightward. Moving rightward means that the table 158 on the bed 170 moves upward in the drawing (or rightward when looking at the bed from the feet of the subject 200).

FIG. 6A illustrates a state in which the operator is not operating (or touching) any operation unit of the console 130 and the table 158 on the bed 170 is at rest. At this point, the plurality of light-emitting devices 300 and 302 are not emitting light.

FIG. 6B illustrates a state in which the operator has touched the first operation unit 132 of the console 130 or the operator has pressed halfway the first operation unit 132 of the console 130. The first operation unit 132 corresponds to an operation of moving the table 158 on the bed 170 rightward.

When the determining unit 118 determines that the operator has touched or pressed halfway the first operation unit 132, the presenting device control unit 114 presents, through the plurality of light-emitting devices 300 and 302 (second presenting device 172), information indicating that the table 158 on the bed 170 is to be moved rightward (i.e., information indicating that rightward movement is scheduled). The presenting device control unit 114 controls the plurality of light-emitting devices 300 and 302 such that light-emitting devices corresponding to the rightward direction emit light. For example, of the plurality of light-emitting devices 300 and 302, six light-emitting devices corresponding to the rightward direction (or on the upper side in the drawing) emit light.

By operating the first operation unit 132 as described above, the operator can recognize, without fully pressing the first operation unit 132, that the table 158 on the bed 170 is to move rightward.

FIG. 6C illustrates a state in which the operator has fully pressed the first operation unit 132 of the console 130. When the determining unit 118 determines that the operator has fully pressed the first operation unit 132, the presenting device control unit 114 presents, through the plurality of light-emitting devices 300 and 302, information indicating that the table 158 on the bed 170 is being moved rightward (i.e., information indicating that rightward movement is in progress). The presenting device control unit 114 controls the plurality of light-emitting devices 300 and 302 such that light-emitting devices corresponding to the rightward direction emit light.

The number of light-emitting devices that emit light when the determining unit 118 determines that the operator has fully pressed the first operation unit 132 is greater than the number of light-emitting devices that emit light when the determining unit 118 determines that the operator has touched or pressed halfway the first operation unit 132. For example, of the plurality of light-emitting devices 300 and 302, ten light-emitting devices corresponding to the rightward direction (or on the upper side in the drawing) emit light.

In the description above, the number of light-emitting devices 300 and 302 emitting light is used to distinguish between information indicating that the table 158 on the bed 170 is to be moved rightward (i.e., information indicating that rightward movement is scheduled) and information indicating that the table 158 on the bed 170 is being moved rightward (i.e., information indicating that rightward movement is in progress). It is simply required that a plurality of light-emitting devices (second presenting device 172) emitting light when the determining unit 118 determines that the operator has touched or pressed halfway the first operation unit 132 and a plurality of light-emitting devices (second presenting device 172) emitting light when the determining unit 118 determines that the operator has fully pressed the first operation unit 132 have different modes of presentation.

Thus, when fully pressing the first operation unit 132, the operator can recognize that the table 158 on the bed 170 is moving rightward (i.e., rightward movement is in progress). The description of the third operation unit 136 will be omitted, as the third operation unit 136 is the same as the first operation unit 132 except that it corresponds to leftward movement.

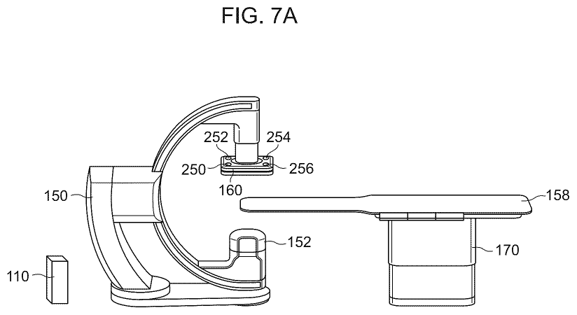

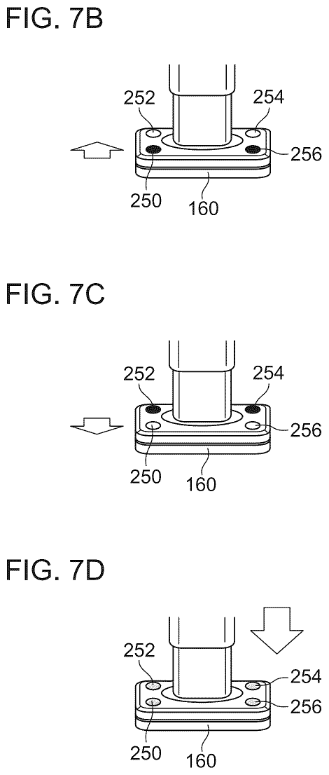

FIG. 7A to FIG. 7D are diagrams each illustrating a mode of presentation related to the radiation detecting unit 160 of the medical imaging system according to the present invention. As illustrated in FIG. 7A, a plurality of light-emitting devices (LEDs) 250, 252, 254, and 256 are installed in the radiation detecting unit 160. The plurality of light-emitting devices 250, 252, 254, and 256 correspond to the first presenting device 154 illustrated in FIG. 3. For example, the plurality of light-emitting devices 250, 252, 254, and 256 are installed at four corners of the radiation detecting unit 160. Light-emitting devices in black are in a non-light-emitting state, and light-emitting devices in white are in a light-emitting state (in a lit state). Light-emitting devices in gray are in a state of repeatedly switching between a light-emitting state and a non-light-emitting state (in a blinking state).

The switching operation units 120 and 122 are operation units for switching the operation target. Here, the operation target is switched from the table 158 on the bed 170 to the radiation detecting unit 160 of the imager 150.

FIG. 7B illustrates a state in which the operator has touched the first operation unit 132 of the console 130 or the operator has pressed halfway the first operation unit 132 of the console 130. The first operation unit 132 corresponds to an operation of moving the radiation detecting unit 160 clockwise (or toward the back in the drawing).

When the determining unit 118 determines that the operator has touched or pressed halfway the first operation unit 132, the presenting device control unit 114 presents, through the plurality of light-emitting devices 250, 252, 254, and 256 (first presenting device 154), information indicating that the radiation detecting unit 160 is to be moved clockwise (i.e., information indicating that clockwise movement is scheduled). The presenting device control unit 114 controls the plurality of light-emitting devices 250, 252, 254, and 256 such that light-emitting devices corresponding to the clockwise direction emit light. For example, the plurality of light-emitting devices 252 and 254 on the back side in the drawing emit light.

By operating the first operation unit 132 as described above, the operator can recognize, without fully pressing the first operation unit 132, that the radiation detecting unit 160 is to be moved clockwise. When fully pressing the first operation unit 132, the operator can move the radiation detecting unit 160 clockwise using the imager movement mechanism 156.

FIG. 7C illustrates a state in which the operator has touched the third operation unit 136 of the console 130 or the operator has pressed halfway the third operation unit 136 of the console 130. The third operation unit 136 corresponds to an operation of moving the radiation detecting unit 160 counterclockwise (or toward the front in the drawing).

When the determining unit 118 determines that the operator has touched or pressed halfway the third operation unit 136, the presenting device control unit 114 presents, through the plurality of light-emitting devices 250, 252, 254, and 256 (first presenting device 154), information indicating that the radiation detecting unit 160 is to be moved counterclockwise (i.e., information indicating that counterclockwise movement is scheduled). The presenting device control unit 114 controls the plurality of light-emitting devices 250, 252, 254, and 256 such that light-emitting devices corresponding to the counterclockwise direction emit light. For example, the plurality of light-emitting devices 250 and 256 on the front side in the drawing emit light.

By operating the third operation unit 136 as described above, the operator can recognize, without fully pressing the third operation unit 136, that the radiation detecting unit 160 is to be moved counterclockwise. When fully pressing the third operation unit 136, the operator can move the radiation detecting unit 160 counterclockwise using the imager movement mechanism 156.

FIG. 7D illustrates a state in which the operator has touched the sixth operation unit 146 of the console 130 or the operator has pressed halfway the sixth operation unit 146 of the console 130. The sixth operation unit 146 corresponds to an operation of moving the radiation detecting unit 160 downward (or toward the subject).

When the determining unit 118 determines that the operator has touched or pressed halfway the sixth operation unit 146, the presenting device control unit 114 presents, through the plurality of light-emitting devices 250, 252, 254, and 256 (first presenting device 154), information indicating that the radiation detecting unit 160 is to be moved downward (i.e., information indicating that downward movement is scheduled). The presenting device control unit 114 controls the plurality of light-emitting devices 250, 252, 254, and 256 such that light-emitting devices corresponding to the downward direction emit light. For example, the plurality of light-emitting devices 250, 252, 254, and 256 blink.

By operating the sixth operation unit 146 as described above, the operator can recognize, without fully pressing the sixth operation unit 146, that the radiation detecting unit 160 is to be moved downward. When fully pressing the sixth operation unit 146, the operator can move the radiation detecting unit 160 downward using the imager movement mechanism 156.

Although modes for moving the radiation detecting unit 160 have been described, the present invention is also applicable to movable components of the imager 150, such as the radiation irradiation unit 152 and the C-arm. When the determining unit 118 determines that the operator has touched or pressed halfway the fifth operation unit 144, the presenting device control unit 114 may present, through the plurality of light-emitting devices 300 and 302 (second presenting device 172), information indicating that the table 158 on the bed 170 is to be moved upward (i.e., information indicating that upward movement is scheduled). The presenting device control unit 114 presents, for example, all the plurality of light-emitting devices 300 and 302 in a lit state.

When the determining unit 118 determines that the operator has touched or pressed halfway the sixth operation unit 146, the presenting device control unit 114 may present, through the plurality of light-emitting devices 300 and 302 (second presenting device 172), information indicating that the table 158 on the bed 170 is to be moved downward (i.e., information indicating that downward movement is scheduled). The presenting device control unit 114 presents, for example, all the plurality of light-emitting devices 300 and 302 in a blinking state.

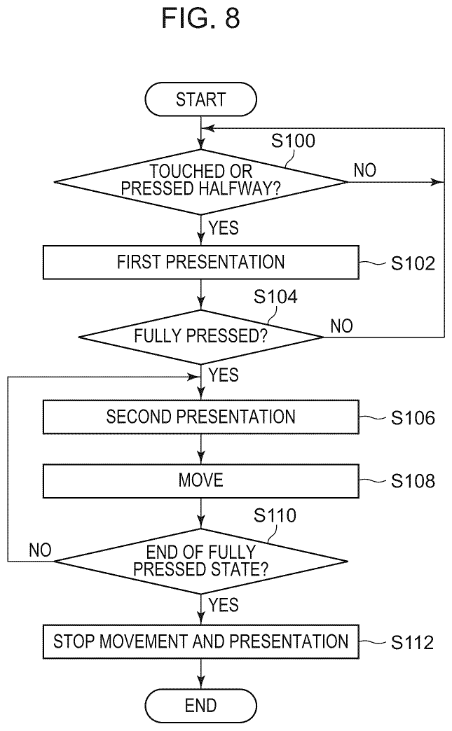

FIG. 8 is a flowchart illustrating an operation of the medical imaging system according to the present invention. Although modes of moving the table 158 on the bed 170 will be described here, other components of the medical imaging system may be moved.

S100: The determining unit 118 determines whether the operator has touched or pressed halfway a predetermined operation unit of the console 130.

S102: When the determining unit 118 determines that the operator has touched or pressed halfway the predetermined operation unit, the presenting device control unit 114 controls the second presenting device 172 to present information indicating that the table 158 on the bed 170 is to be moved in a predetermined direction (first presentation). At this point, the table 158 on the bed 170 is at rest.

S104: The determining unit 118 determines whether the operator has fully pressed the predetermined operation unit.

S106: When the determining unit 118 determines that the operator has fully pressed the predetermined operation unit, the presenting device control unit 114 controls the second presenting device 172 to present, around the bed 170 or on the table 158 on the bed 170, information indicating that the table 158 on the bed 170 is being moved in the predetermined direction (second presentation). The second presentation is different (or distinguishable) from the first presentation.

S108: When the determining unit 118 determines that the operator has fully pressed the predetermined operation unit, the movement control unit 116 performs control to move the table 158 on the bed 170 in the predetermined direction using the bed movement mechanism 174.

S110: When there is a transition from the state of determining that the operator has fully pressed the predetermined operation unit to the state of determining that the operator no longer fully presses the predetermined operation unit, the determining unit 118 determines that the fully-pressed state of the predetermined operation unit has ended.

S112: When the determining unit 118 determines that the fully-pressed state of the predetermined operation unit has ended, the presenting device control unit 114 stops presenting the information indicating that the table 158 on the bed 170 is being moved in the predetermined direction (second presentation) presented through the second presenting device 172. Then, the movement control unit 116 stops the movement of the table 158 on the bed 170 that has been moved in the predetermined direction by the bed movement mechanism 174.

The medical imaging system according to the present invention includes the bed 170 on which the subject is placed; the console 130 including a plurality of operation units and configured to allow a predetermined operation unit to be operated to give an instruction to move the table 158 on the bed 170 in a movement direction corresponding to the predetermined operation unit; the movement mechanism (bed movement mechanism 174) configured to move the table 158 on the bed 170 on the basis of operation information of the predetermined operation unit; the determining unit 118 configured to determine whether the operator has touched the predetermined operation unit; and the presenting unit (second presenting device 172) configured to present, around the bed 170 or on the table 158 on the bed 170, information about the movement direction corresponding to the predetermined operation unit when the determining unit 118 determines that the operator has touched the predetermined operation unit.

The medical imaging system may include the determining unit 118 configured to determine whether the operator has pressed a predetermined operation unit halfway; and the presenting unit (second presenting device 172) configured to present, around the bed 170 or on the table 158 on the bed 170, information about the movement direction corresponding to the predetermined operation unit when the determining unit 118 determines that the operator has pressed the predetermined operation unit halfway.

The medical imaging system according to the present invention includes the imager 150 having the function of imaging the subject; the console 130 including a plurality of operation units and configured to allow a predetermined operation unit to be operated to give an instruction to move the component 160 or 152 of the imager 150 in a movement direction corresponding to the predetermined operation unit; the movement mechanism configured to move the component 160 or 152 of the imager 150 on the basis of operation information of the predetermined operation unit; the determining unit 118 configured to determine whether the operator has touched the predetermined operation unit; and the presenting unit (first presenting device 154) configured to present, on the component 160 or 152 of the imager 150 or around the imager 150, information about the movement direction corresponding to the predetermined operation unit when the determining unit 118 determines that the operator has touched the predetermined operation unit.

The medical imaging system may include the determining unit 118 configured to determine whether the operator has pressed a predetermined operation unit halfway; and the presenting unit (first presenting device 154) configured to present, on the component 160 or 152 of the imager 150 or around the imager 150, information about the movement direction corresponding to the predetermined operation unit when the determining unit 118 determines that the operator has pressed the predetermined operation unit halfway.

### Embodiment 2

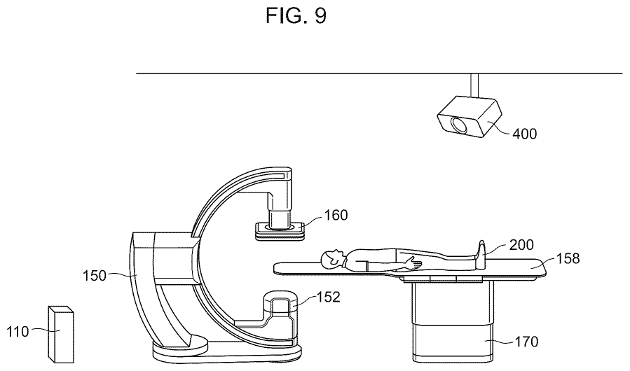

Embodiment 2 of the medical imaging system according to the present invention will be described with reference to FIG. 9 to FIG. 14C. Embodiment 2 differs from Embodiment 1 in that a presenting device 400 performs presentation around the bed 170 or on the table 158 on the bed 170 by using a projecting technique of projecting an image related to a movement direction.

FIG. 9 is a diagram illustrating a configuration of the medical imaging system according to the present embodiment. The presenting device 400 is installed on the ceiling of the imaging room. The presenting device 400 is a projector configured to display a predetermined image by projecting it, for example, onto the table 158 on the bed 170, or onto the floor surface of the imaging room. Therefore, the projection direction of the presenting device 400 is toward the table 158 on the bed 170 or toward the floor surface of the imaging room. The presenting device 400 is connected to the controller 110. The other components in FIG. 9 will not be described here, as they are the same as those in FIG. 1A and FIG. 1B.

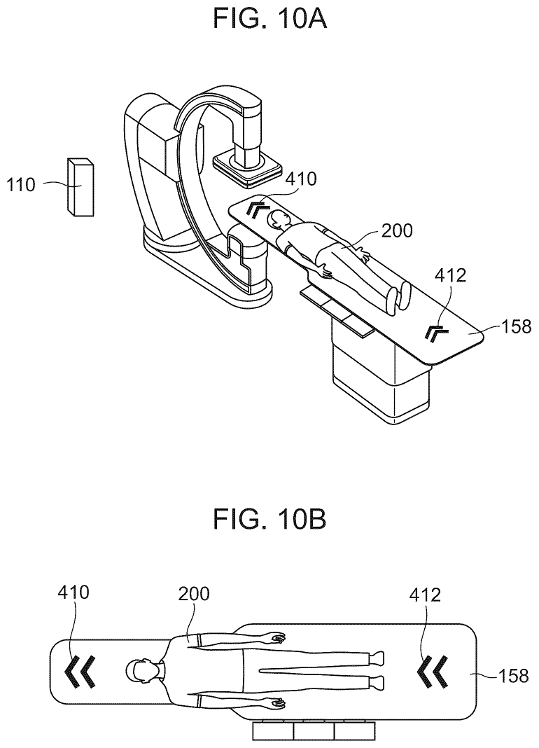

FIG. 10A and FIG. 10B are diagrams illustrating an exemplary mode of presentation of the presenting device 400 of the medical imaging system according to the present embodiment. FIG. 10A is a perspective view, and FIG. 10B is a top view.

Images projected by the presenting device 400 installed on the ceiling of the imaging room are displayed as images 410 and 412. The presenting device 400 projects the images 410 and 412 at positions avoiding the subject 200. For example, the images 410 and 412 are projected onto end portions of the table 158 on the bed 170. The presenting device 400 may have the function of recognizing the position of the subject 200 and the function of recognizing the position of the head of the subject 200, and can project the images 410 and 412 at positions avoiding the position of the subject 200 (head). Specifically, the function of recognizing the position of the subject 200 involves image recognition on an image captured, for example, using a visible light camera or an infrared camera. In the image captured using the camera, the presenting device 400 recognizes the position of the head or body of the subject 200. The position of the head can be recognized by using a landmark, such as eyes, nose, or mouth, as a reference. The position of the body of the subject 200 can be recognized by using a landmark, such as a torso, head, feet, or hands, as a reference. The image recognition may involve matching using a template. The presenting device 400 may estimate the body temperature in an infrared image of the subject 200 and recognize the position of the head or body of the subject 200.

Then, the presenting device 400 projects the images 410 and 412 at positions avoiding the position of the head or body of the subject 200 recognized as described above. Specifically, the presenting device 400 sets a projection direction, a projection position, a projection region, and the like on the basis of the position of the head or body of the subject 200 such that an image is not projected onto the head or body of the subject 200.

The presenting device 400 projects the images 410 and 412 here, but may project one of them.

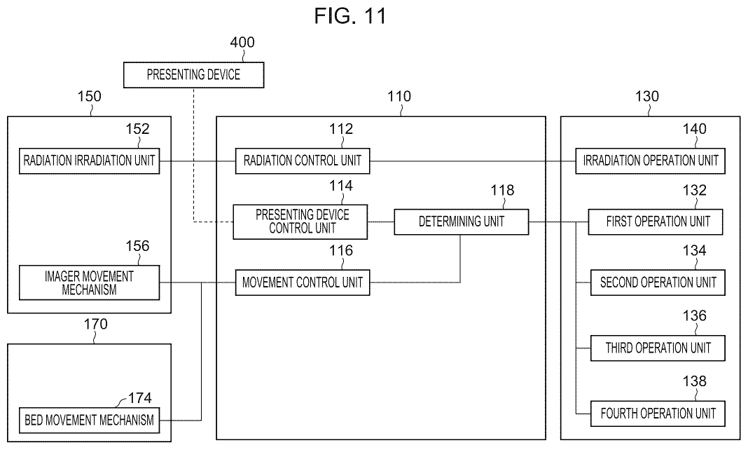

FIG. 11 is a diagram illustrating an exemplary configuration of the medical imaging system according to the present invention. The medical imaging system illustrated in FIG. 11 includes the presenting device 400 capable of projecting a predetermined image. The presenting device 400 is connected to the presenting device control unit 114. The presenting device control unit 114 has the function of controlling the presenting device 400 configured to project images related to the movement direction of the imager 150 and the bed 170. The other components in FIG. 11 will not be described here, as they are the same as those in FIG. 3.

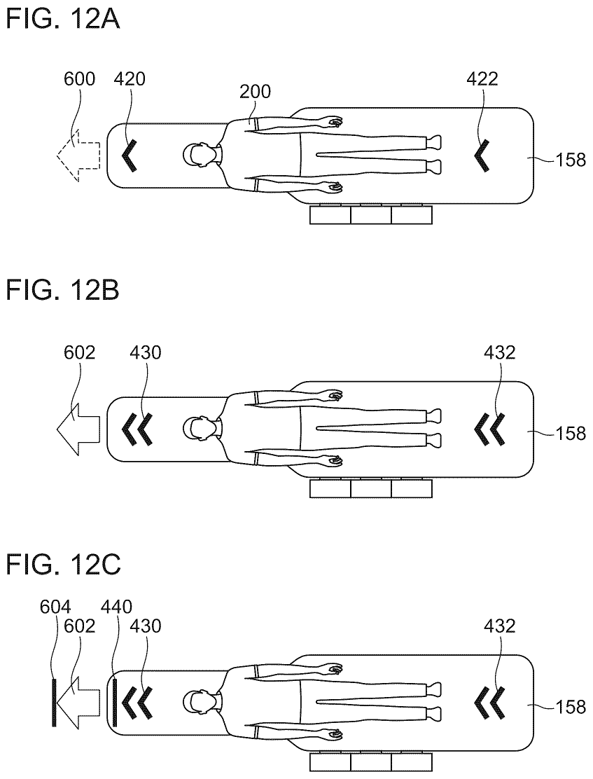

FIG. 12A to FIG. 12C each illustrate a mode in which the table 158 on the bed 170 moves forward. Moving forward means that the table 158 on the bed 170 moves leftward in the drawing (or toward the head of the subject 200).

FIG. 12A illustrates a state in which the operator has touched the second operation unit 134 of the console 130 or the operator has pressed halfway the second operation unit 134 of the console 130. The second operation unit 134 corresponds to an operation of moving the table 158 on the bed 170 forward.

When the determining unit 118 determines that the operator has touched or pressed halfway the second operation unit 134, the presenting device control unit 114 presents, through the presenting device 400, information indicating that the table 158 on the bed 170 is to be moved forward (i.e., information indicating that forward movement is scheduled). The presenting device control unit 114 controls the presenting device 400 to project images 420 and 422 corresponding to the forward direction onto the table 158 on the bed 170. For example, the images 420 and 422 indicate the forward direction (or the leftward direction in the drawing).

By operating the second operation unit 134 as described above, the operator can recognize, without fully pressing the second operation unit 134, that the table 158 on the bed 170 is to move forward. Note that when the operator is not operating (or touching) any operation unit of the console 130 and the table 158 on the bed 170 is at rest, no image is projected onto the table 158 on the bed 170.

FIG. 12B illustrates a state in which the operator has fully pressed the second operation unit 134 of the console 130. When the determining unit 118 determines that the operator has fully pressed the second operation unit 134, the presenting device control unit 114 presents, through the presenting device 400, information indicating that the table 158 on the bed 170 is being moved forward (i.e., information indicating that forward movement is in progress). The presenting device control unit 114 controls the presenting device 400 to project images 430 and 432 corresponding to the forward direction onto the table 158 on the bed 170.

The number of marks in the images 430 and 432 projected when the determining unit 118 determines that the operator has fully pressed the second operation unit 134 is greater than the number of marks in the images 420 and 422 projected when the determining unit 118 determines that the operator has touched or pressed halfway the second operation unit 134. For example, the images 420 and 422 each include one mark, whereas the images 430 and 432 each include two marks.

In the description above, the number of marks in the images is used to distinguish between information indicating that the table 158 on the bed 170 is to be moved forward (i.e., information indicating that forward movement is scheduled) and information indicating that the table 158 on the bed 170 is being moved forward (i.e., information indicating that forward movement is in progress). It is simply required that the image projected when the determining unit 118 determines that the operator has touched or pressed halfway the second operation unit 134 and the image projected when the determining unit 118 determines that the operator has fully pressed the second operation unit 134 have different modes of presentation.

Thus, when fully pressing the second operation unit 134, the operator can recognize that the table 158 on the bed 170 is moving forward (i.e., forward movement is in progress).

FIG. 12C illustrates a state in which the operator has fully pressed the second operation unit 134 of the console 130, and indicates that the table 158 on the bed 170 is near the limit of forward movement. The presenting device control unit 114 presents, through the presenting device 400, information indicating that the table 158 on the bed 170 is being moved forward and is near the limit of movement (i.e., information indicating that forward movement is in progress and the limit of the movement is near). The presenting device control unit 114 controls the presenting device 400 to project an image 440 indicating that the limit of movement is near. For example, the image 440 includes a bar-shaped mark.

By checking the image 440 indicating that the limit of movement is near, the operator can recognize that the table 158 on the bed 170 is being moved forward and that the limit of movement is near.

As illustrated in FIG. 12A, the presenting device control unit 114 controls the presenting device 400 to present, around the bed 170, information indicating that the table 158 on the bed 170 is to be moved forward (i.e., information indicating that forward movement is scheduled). Specifically, the presenting device control unit 114 controls the presenting device 400 to project, onto the floor surface near the bed 170, an image 600 corresponding to the forward direction. For example, the image 600 is drawn in a broken line and indicates the forward direction (or the leftward direction in the drawing). As illustrated in FIG. 12B and FIG. 12C, the presenting device control unit 114 controls the presenting device 400 to present, around the bed 170, information indicating that the table 158 on the bed 170 is being moved forward (i.e., information indicating that forward movement is in progress). Specifically, the presenting device control unit 114 controls the presenting device 400 to project, around the bed 170, an image 602 corresponding to the forward direction. For example, the image 602 is drawn in a solid line to be distinguished from the image 600 drawn in a broken line, and indicates the forward direction (or the leftward direction in the drawing). The presenting device control unit 114 also controls the presenting device 400 to project an image 604 indicating that the limit of movement of the table 158 is near.

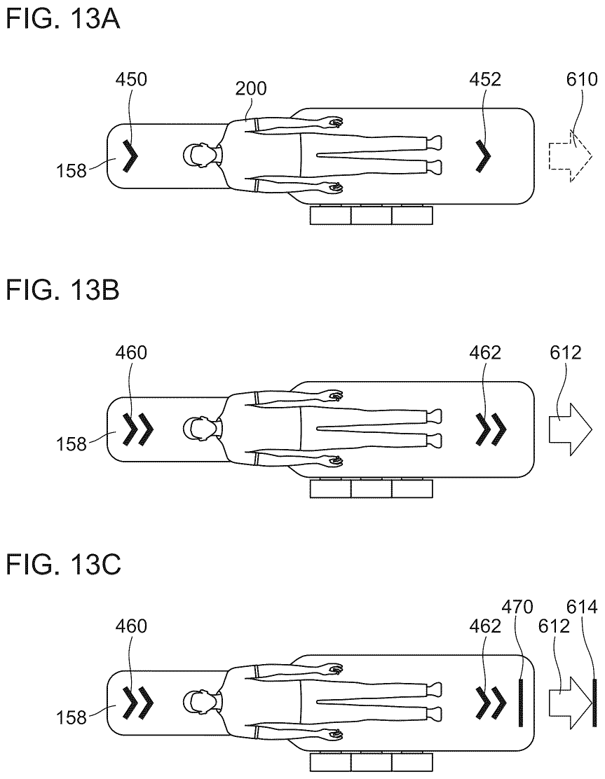

FIG. 13A to FIG. 13C each illustrate a mode in which the table 158 on the bed 170 moves backward. Moving backward means that the table 158 on the bed 170 moves rightward in the drawing (or in the direction of the feet of the subject 200).

FIG. 13A illustrates a state in which the operator has touched the fourth operation unit 138 of the console 130 or the operator has pressed halfway the fourth operation unit 138 of the console 130. The fourth operation unit 138 corresponds to an operation of moving the table 158 on the bed 170 backward.

When the determining unit 118 determines that the operator has touched or pressed halfway the fourth operation unit 138, the presenting device control unit 114 presents, through the presenting device 400, information indicating that the table 158 on the bed 170 is to be moved backward (i.e., information indicating that backward movement is scheduled). The presenting device control unit 114 controls the presenting device 400 to project images 450 and 452 corresponding to the backward direction onto the table 158 on the bed 170. For example, the images 450 and 452 indicate the backward direction (or the rightward direction in the drawing).

By operating the fourth operation unit 138 as described above, the operator can recognize, without fully pressing the fourth operation unit 138, that the table 158 on the bed 170 is to move backward.

FIG. 13B illustrates a state in which the operator has fully pressed the fourth operation unit 138 of the console 130. When the determining unit 118 determines that the operator has fully pressed the fourth operation unit 138, the presenting device control unit 114 presents, through the presenting device 400, information indicating that the table 158 on the bed 170 is being moved backward (i.e., information indicating that backward movement is in progress). The presenting device control unit 114 controls the presenting device 400 to project images 460 and 462 corresponding to the backward direction onto the table 158 on the bed 170.

The number of marks in the images 460 and 462 projected when the determining unit 118 determines that the operator has fully pressed the fourth operation unit 138 is greater than the number of marks in the images 450 and 452 projected when the determining unit 118 determines that the operator has touched or pressed halfway the fourth operation unit 138. For example, the images 450 and 452 each include one mark, whereas the images 460 and 462 each include two marks.

In the description above, the number of marks in the images is used to distinguish between information indicating that the table 158 on the bed 170 is to be moved backward (i.e., information indicating that backward movement is scheduled) and information indicating that the table 158 on the bed 170 is being moved backward (i.e., information indicating that backward movement is in progress). It is simply required that the image projected when the determining unit 118 determines that the operator has touched or pressed halfway the fourth operation unit 138 and the image projected when the determining unit 118 determines that the operator has fully pressed the fourth operation unit 138 have different modes of presentation.

Thus, when fully pressing the fourth operation unit 138, the operator can recognize that the table 158 on the bed 170 is moving backward (i.e., backward movement is in progress).

FIG. 13C illustrates a state in which the operator has fully pressed the fourth operation unit 138 of the console 130, and indicates that the table 158 on the bed 170 is near the limit of backward movement. The presenting device control unit 114 presents, through the presenting device 400, information indicating that the table 158 on the bed 170 is being moved backward and is near the limit of movement (i.e., information indicating that backward movement is in progress and the limit of the movement is near). The presenting device control unit 114 controls the presenting device 400 to project an image 470 indicating that the limit of movement is near. For example, the image 470 includes a bar-shaped mark.

By checking the image 470 indicating that the limit of movement is near, the operator can recognize that the table 158 on the bed 170 is being moved backward and that the limit of movement is near.

As illustrated in FIG. 13A, the presenting device control unit 114 controls the presenting device 400 to present, around the bed 170, information indicating that the table 158 on the bed 170 is to be moved backward (i.e., information indicating that backward movement is scheduled). Specifically, the presenting device control unit 114 controls the presenting device 400 to project, onto the floor surface near the bed 170, an image 610 corresponding to the backward direction. For example, the image 610 is drawn in a broken line and indicates the backward direction (or the rightward direction in the drawing). As illustrated in FIG. 13B and FIG. 13C, the presenting device control unit 114 controls the presenting device 400 to present, around the bed 170, information indicating that the table 158 on the bed 170 is being moved backward (i.e., information indicating that backward movement is in progress). Specifically, the presenting device control unit 114 controls the presenting device 400 to project, around the bed 170, an image 612 corresponding to the backward direction. For example, the image 612 is drawn in a solid line to be distinguished from the image 610 drawn in a broken line, and indicates the backward direction (or the rightward direction in the drawing). The presenting device control unit 114 also controls the presenting device 400 to project an image 614 indicating that the limit of movement of the table 158 is near.

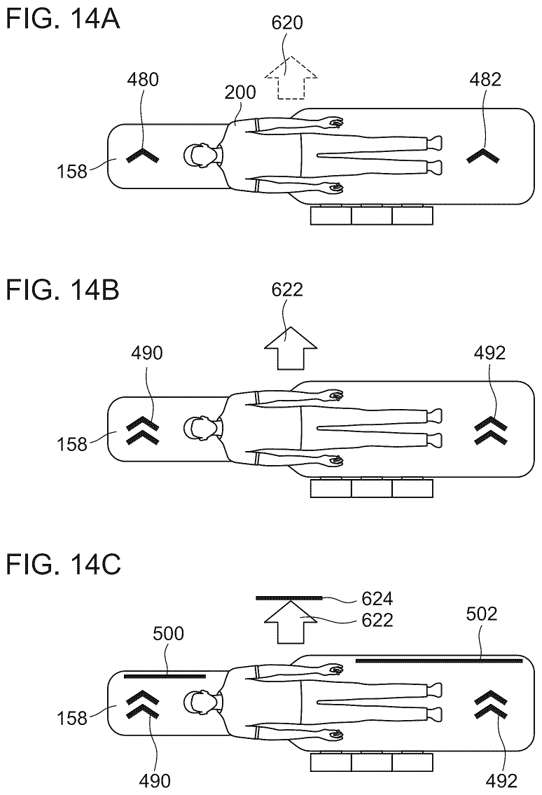

FIG. 14A to FIG. 14C each illustrate a mode in which the table 158 on the bed 170 moves rightward. Moving rightward means that the table 158 on the bed 170 moves upward in the drawing (or rightward when looking at the bed from the feet of the subject 200).

FIG. 14A illustrates a state in which the operator has touched the first operation unit 132 of the console 130 or the operator has pressed halfway the first operation unit 132 of the console 130. The first operation unit 132 corresponds to an operation of moving the table 158 on the bed 170 rightward.

When the determining unit 118 determines that the operator has touched or pressed halfway the first operation unit 132, the presenting device control unit 114 presents, through the presenting device 400, information indicating that the table 158 on the bed 170 is to be moved rightward (i.e., information indicating that rightward movement is scheduled). The presenting device control unit 114 controls the presenting device 400 to project images 480 and 482 corresponding to the rightward direction onto the table 158 on the bed 170. For example, the images 480 and 482 indicate the rightward direction (or the upward direction in the drawing).

By operating the first operation unit 132 as described above, the operator can recognize, without fully pressing the first operation unit 132, that the table 158 on the bed 170 is to move rightward.

FIG. 14B illustrates a state in which the operator has fully pressed the first operation unit 132 of the console 130. When the determining unit 118 determines that the operator has fully pressed the first operation unit 132, the presenting device control unit 114 presents, through the presenting device 400, information indicating that the table 158 on the bed 170 is being moved rightward (i.e., information indicating that rightward movement is in progress). The presenting device control unit 114 controls the presenting device 400 to project images 490 and 492 corresponding to the rightward direction onto the table 158 on the bed 170.

The number of marks in the images 490 and 492 projected when the determining unit 118 determines that the operator has fully pressed the first operation unit 132 is greater than the number of marks in the images 480 and 482 projected when the determining unit 118 determines that the operator has touched or pressed halfway the first operation unit 132. For example, the images 480 and 482 each include one mark, whereas the images 490 and 492 each include two marks.

In the description above, the number of marks in the images is used to distinguish between information indicating that the table 158 on the bed 170 is to be moved rightward (i.e., information indicating that rightward movement is scheduled) and information indicating that the table 158 on the bed 170 is being moved rightward (i.e., information indicating that rightward movement is in progress). It is simply required that the image projected when the determining unit 118 determines that the operator has touched or pressed halfway the first operation unit 132 and the image projected when the determining unit 118 determines that the operator has fully pressed the first operation unit 132 have different modes of presentation.

Thus, when fully pressing the first operation unit 132, the operator can recognize that the table 158 on the bed 170 is moving rightward (i.e., rightward movement is in progress).

FIG. 14C illustrates a state in which the operator has fully pressed the first operation unit 132 of the console 130, and indicates that the table 158 on the bed 170 is near the limit of rightward movement. The presenting device control unit 114 presents, through the presenting device 400, information indicating that the table 158 on the bed 170 is being moved rightward and is near the limit of movement (i.e., information indicating that rightward movement is in progress and the limit of the movement is near). The presenting device control unit 114 controls the presenting device 400 to project images 500 and 502 indicating that the limit of movement is near. For example, the images 500 and 502 each include a bar-shaped mark.

By checking the images 500 and 502 indicating that the limit of movement is near, the operator can recognize that the table 158 on the bed 170 is being moved rightward and that the limit of movement is near. The description of the third operation unit 136 will be omitted, as the third operation unit 136 is the same as the first operation unit 132 except that it corresponds to leftward movement.

As illustrated in FIG. 14A, the presenting device control unit 114 controls the presenting device 400 to present, around the bed 170, information indicating that the table 158 on the bed 170 is to be moved rightward (i.e., information indicating that rightward movement is scheduled). Specifically, the presenting device control unit 114 controls the presenting device 400 to project, onto the floor surface near the bed 170, an image 620 corresponding to the rightward direction. For example, the image 620 is drawn in a broken line and indicates the rightward direction (or the upward direction in the drawing). As illustrated in FIG. 14B and FIG. 14C, the presenting device control unit 114 controls the presenting device 400 to present, around the bed 170, information indicating that the table 158 on the bed 170 is being moved rightward (i.e., information indicating that rightward movement is in progress). Specifically, the presenting device control unit 114 controls the presenting device 400 to project, around the bed 170, an image 622 corresponding to the rightward direction. For example, the image 622 is drawn in a solid line to be distinguished from the image 620 drawn in a broken line, and indicates the rightward direction (or the upward direction in the drawing). The presenting device control unit 114 also controls the presenting device 400 to project an image 624 indicating that the limit of movement of the table 158 is near.

The presenting device control unit 114 can control the presenting device 400 to project an image around an operation unit of the console 130. This allows the operator to recognize the direction in which the table 158 on the bed 170 moves.

The presenting device control unit 114 can control the presenting device 400 to project, onto the floor surface near the bed 170, an image indicating a region (danger area) corresponding to the movable range of the table 158 on the bed 170. The movable range corresponds to the limit of movement described above. The operator can thus recognize the movement direction and the movable range of the table 158 on the bed 170.

The medical imaging system includes a radiation imaging device (e.g., radiation irradiation unit, radiation detecting unit) in the description above, but may include an MRI machine or a CT scanner. The medical imaging system is simply required to include, for example, the controller 110, the console 130, and the bed 170 and may include any type of imaging device.

A computer program that implements the presenting functions of Embodiments 1 and 2 can be executed by supplying it to a computer through a network or a storage medium (not illustrated). The computer program is a program for causing a computer to execute the presenting methods described above. That is, the computer program is a program for implementing the functions of the medical imaging system on a computer. A storage medium can store the computer program.

The present invention can present, in advance, information about a movement direction corresponding to a predetermined operation unit of the console.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A medical imaging system comprising:
a bed on which a subject is placed;
a console including a plurality of operation units and configured to allow a predetermined operation unit to be operated to give an instruction to move a table on the bed in a movement direction corresponding to the predetermined operation unit;
a movement mechanism configured to move the table on the bed on the basis of operation information of the predetermined operation unit;
a determining unit configured to determine whether an operator has touched the predetermined operation unit; and
a presenting unit configured to present, around the bed or on the table on the bed, information about the movement direction corresponding to the predetermined operation unit when the determining unit determines that the operator has touched the predetermined operation unit.

2. The medical imaging system according to claim 1, wherein the determining unit is capable of distinguishing between a state in which the operator has touched the predetermined operation unit and a state in which the operator has fully pressed the predetermined operation unit.

3. The medical imaging system according to claim 2, wherein the plurality of operation units each include a contact sensor configured to detect a state of being touched by the operator, and a press sensor configured to detect a state of being fully pressed by the operator.

4. The medical imaging system according to claim 2, wherein when the determining unit determines that the operator has fully pressed the predetermined operation unit, the presenting unit presents, around the bed or on the table on the bed, information indicating that the table on the bed is being moved in the movement direction, and the movement mechanism moves the table on the bed in the movement direction.

5. The medical imaging system according to claim 1, wherein the presenting unit includes a plurality of light-emitting devices.

6. The medical imaging system according to claim 5, wherein the plurality of light-emitting devices are installed on the table on the bed, and the medical imaging system comprises a controller configured to perform control such that a light-emitting device corresponding to the movement direction emits light.

7. The medical imaging system according to claim 1, wherein the presenting unit is configured to perform presentation using a projecting technique in which an image related to the movement direction is projected around the bed or onto the table on the bed.

8. The medical imaging system according to claim 7, wherein the presenting unit has a function of recognizing a position of the subject, and is configured to project the image related to the movement direction at a position avoiding the position of the subject.

9. The medical imaging system according to claim 1, wherein the presenting unit has different modes of presentation to allow a distinction between when the determining unit determines that the operator has touched the predetermined operation unit and when the determining unit determines that the operator has fully pressed the predetermined operation unit.

10. The medical imaging system according to claim 1, wherein the presenting unit is configured to present information indicating that a limit of movement is near.

11. A medical imaging system comprising:
a bed on which a subject is placed;
a console including a plurality of operation units and configured to allow a predetermined operation unit to be operated to give an instruction to move a table on the bed in a movement direction corresponding to the predetermined operation unit;
a movement mechanism configured to move the table on the bed on the basis of operation information of the predetermined operation unit;
a determining unit configured to determine whether an operator has pressed the predetermined operation unit halfway; and
a presenting unit configured to present, around the bed or on the table on the bed, information about the movement direction corresponding to the predetermined operation unit when the determining unit determines that the operator has pressed the predetermined operation unit halfway.

12. The medical imaging system according to claim 11, wherein the determining unit is configured to distinguish between a state in which the operator has pressed the predetermined operation unit halfway and a state in which the operator has fully pressed the predetermined operation unit.

13. The medical imaging system according to claim 11, wherein the plurality of operation units each include a press sensor configured to detect a state of being pressed halfway by the operator and a state of being fully pressed by the operator.

14. The medical imaging system according to claim 12, wherein when the determining unit determines that the operator has fully pressed the predetermined operation unit, the presenting unit presents, around the bed or on the table on the bed, information indicating that the table on the bed is being moved in the movement direction, and the movement mechanism moves the table on the bed in the movement direction.

15. A medical imaging system comprising:
an imager having a function of imaging a subject;
a console including a plurality of operation units and configured to allow a predetermined operation unit to be operated to give an instruction to move a component of the imager in a movement direction corresponding to the predetermined operation unit;
a movement mechanism configured to move the component of the imager on the basis of operation information of the predetermined operation unit;
a determining unit configured to determine whether an operator has touched the predetermined operation unit; and
a presenting unit configured to present, on the component of the imager or around the imager, information about the movement direction corresponding to the predetermined operation unit when the determining unit determines that the operator has touched the predetermined operation unit.

16. A medical imaging system comprising:
an imager having a function of imaging a subject;
a console including a plurality of operation units and configured to allow a predetermined operation unit to be operated to give an instruction to move a component of the imager in a movement direction corresponding to the predetermined operation unit;
a movement mechanism configured to move the component of the imager on the basis of operation information of the predetermined operation unit;
a determining unit configured to determine whether an operator has pressed the predetermined operation unit halfway; and
a presenting unit configured to present, on the component of the imager or around the imager, information about the movement direction corresponding to the predetermined operation unit when the determining unit determines that the operator has pressed the predetermined operation unit halfway.
